(19) United States
(12) Patent Application Publication
(10) Pub. No.: US 2014/0037718 A1
(43) Pub. Date: Feb. 6, 2014

(54) TRANSDERMAL PAIN GEL

(71) Applicant: George W. Lutz, Warren, NJ (US)

(72) Inventor: George W. Lutz, Warren, NJ (US)

(21) Appl. No.: 14/047,446

(22) Filed: Oct. 7, 2013

Related U.S. Application Data
(63) Continuation-in-part of application No. 12/718,417, filed on Mar. 5, 2010.
(60) Provisional application No. 61/158,161, filed on Mar. 6, 2009.

(51) Int. Cl. A61K 9/127; A61K 31/197; A61K 31/167; A61K 31/135; A61K 31/192

(52) U.S. Cl. CPC A61K 9/127; A61K 31/135; A61K 31/192; A61K 31/167; A61K 31/197
USPC 424/450; 514/646; 514/567

(57) ABSTRACT

A transdermal gel including a complementary array of medicinal components has beneficial effects for pain relief in muscular and connective tissues. The medicinal components include active ingredients having a synergistic effect for permitting musculoskeletal movement by countering the symptoms of musculoskeletal pain and being non-narcotic for avoiding dependency, and are combined in a liposomal base with a wetting agent to form a gel consistency suitable for skin application. The transdermal gel allows topical application of greater quantities and concentrations of the active ingredients than could safely be obtained via conventional oral administration.

### CROSS REFERENCE TO RELATED APPLICATIONS

This patent application is a Continuation-in-Part (CIP) of application Ser. No. 12/718,417, filed Mar. 5, 2010, entitled “TRANSDERMAL PAIN GEL”, and claims the benefit under 35 U.S.C. §119(e) of U.S. Provisional Patent Application No. 61/158,161 filed on Mar. 6, 2009, entitled, “TRANSDERMAL PAIN GEL,” the contents and teachings of which are hereby incorporated by reference in their entirety.

### BACKGROUND

Pain medication relieves unpleasant symptoms caused by physiological responses to injury or other perceived stimuli to actual or potential human tissue damage. Most often, pain medication and relief strives to block neurotransmitters which transmit pain stimuli to the brain that results in the uncomfortable pain sensations experienced by the afflicted person. Although human evolution has provided such pain recognition and the accompanying uncomfortable sensation as a means of self preservation, to inform the body of eminent possible tissue damage, once the initial pain sensation alerts the human subject, subsequent stimuli merely prolong the uncomfortable nature of the pain responsive neurotransmitters.

Neurotransmitters cause transmissions along nerve fibers comprised of neurons (nerve cells) that transmit minute electrical signals. Individual neurons transmit across a synapse defined by an intercell gap between individual neurons. The neurotransmitters travel in the synapse between individual neurons to transmit the pain stimuli, or signal. Thus, preventing neurotransmitters from crossing the synapse prevents transmission of pain stimuli. Typical pain medications deliver substances that interrupt the neurotransmitter path between neurons to avoid the painful sensation perceived by the human subject.

In particular, so-called glutamate transmitters are responsive at the spinal cord level and thus are often associated with chronic pain response. Glutamate is a neurotransmitter triggering spinal excitatory synaptic transmission and for generation and maintenance of spinal states of pain hypersensitivity via activation of glutamate receptors. The glutamate transporter system is the primary mechanism for the inactivation of synaptically released glutamate and the maintenance of glutamate homeostasis. Recent studies raise the suggestion that spinal glutamate transporter inhibition relieves pathological pain, suggesting that the spinal glutamate transporter might serve as a therapeutic target for treatment of pathological pain. In addition to its essential metabolic role, glutamate is a major mediator of excitatory signals in the central nervous system and is involved in many physiologic and pathologic processes, including chronic pain. Glutamate exerts its signaling role by acting on glutamate receptors, including N-methyl-D-aspartate (NMDA) receptors. These NMDA receptors are located on the pre- and post-synaptic membranes, as well as, at extra-synaptic sites. Glutamate concentration in the synapse therefore affects the extents of receptor stimulation and excitatory synaptic transmission. One cause of NMDA pain is that protracted activation of small primary afferent fibers can induce states of facilitated spinal sensory processing.

### SUMMARY

Pain medications provide relief by suppressing the nervous system of a patient, and typically operate by deactivating pain sensing mechanisms such that the pain impulses transported by the central nervous system (CNS) are not perceived by the patient. General anesthesia, often administered during surgery, incapacitates the entire CNS of a patient. Less invasive means, such as local anesthesia, are directed to selective immobilization or deactivation of certain areas, such as novocain administered during dental procedures. Often, pain medication disrupts the flow of chemical neurotransmitters in the synapse between individual neurons (nerve cells) that comprise the nerve fibers transmitting the pain impulses.

Configurations herein are based, in part, on the observation that conventional pain medication therapy may encounter difficulty focusing effective and appropriate dosage quantities to an afflicted area. Conventional approaches employ either oral or intravenous delivery mediums which disperse medication throughout the patient's body, overbroadening the delivery area while delivering insufficient quantities to the afflicted area, and also possibly causing undesirable side effects. Further, narcotic based approaches, while providing adequate relief, may cause undesirable side effects such as sleepiness, loss of coordination, and dependency. It would be beneficial to provide a focused pain relief in a synergistic combination of components that delivers appropriate concentrations to an afflicted area to relieve pain, while reducing inflammation and allowing flexibility for permitting movement in the afflicted area. Accordingly, configurations herein provide a transdermal pain gel suitable for topical application and transdermal absorption directly to the afflicted area, such that transdermal absorption delivers medicinal components for suppressing NDMA receptors that otherwise transmit chronic pain stimuli triggering spinal cord sensitization. Substances that interfere and slow down or prevent the NMDA synapse transmission are so-called antagonists, and include ketamine and dextromethorphan. The symbiotic effect of medicinal components (components) for reduced inflammation and increased flexibility permit less restriction of movement while avoiding the side effects and general dexterous compromise of more general anesthetics.

Conventional pain medications suffer from the shortcoming that insufficient quantities of medication are directed to the afflicted areas. Oral administration of pain medication may require excessive doses in order to deliver an effective dose to the afflicted area, thus rendering oral ingestion insufficiently focused. Configurations disclosed herein substantially overcome the shortcomings of conventional pain relief by combining a complementary array of active ingredients, each of which provides a particular aspect of pain relief. The method combines active ingredients, detailed further below, in a liposomal base or other suitable medium, with a wetting agent to produce a creamlike gel suitable for application on the skin over a pain afflicted area. The suspension properties of the gel maintain the active ingredients in communication with the skin so as to provide transdermal absorption through the skin directly to the affected tissue areas, thus avoiding gastrointestinal ingestion and accompanying side effects of circulatory (bloodstream) delivery of the active ingredients.

A transdermal gel or ointment as disclosed further below includes a complementary array of active ingredients has beneficial effects for pain relief in muscular and connective tissues. The active ingredients are combined in a liposomal base with a wetting agent to form a gel or pastelike consistency suitable for skin application. The transdermal gel allows topical application of greater quantities and concentrations of the active ingredients than could safely be obtained via conventional oral administration.

The treatment method includes particular applications to the joint areas of shoulder and knees, particularly due to the relatively high concentration of skeletal structures relative to soft tissue.

Particular configurations recognize the capability of Ketamine as an antidepressant, which contributes to the synergistic effects on the metal and physiological factors in patient treatment.

Topical application on an external skin surface therefore delivers the medication to the afflicted area in a manner and quantity which would may be unsafe and/or impractical to deliver orally or intravenously to achieve a target dosing concentration. The disclosed transdermal gel includes medicinal components for achieving pain relief (via pain receptor blocking), an anti-inflamatory to prevent swelling, and a relaxant component having anti-spasmodic properties, thus achieving a synergistic combination and concentration of medicine focused on an afflicted area in a manner which is unachievable by oral or IV administration. The dosage quantity delivered via the transdermal delivery medium is typically of a prescriptive strength of one or more of the medicinal components, and is thus unavailable in an OTC manner.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a diagram of a neural synapse employing NMDA receptors responsive to the present invention; and

FIG. 2 is an anatomical cross section showing an epidermal region suitable for use with the transdermal gel of the present invention.

### DETAILED DESCRIPTION

A major aspect of pain medication administration, and indeed an underpinning of the medical field of anesthesiology, is that chemical regulation of the CNS can have serious consequences, and the invasiveness of procedures should be carefully scrutinized. Pain relief can generally be categorized along 3 tiers of invasiveness. A first tier includes non-prescription over the counter (OTC) remedies, such as acetaminophen. A second tier includes so-called NSAIDS (nonsteroidal anti-inflammatory drugs), such as aspirin and ibuprofen, also available OTC. Prescription based remedies also include these groups, typically at increased dosage levels. Beyond this, a third group includes narcotic based remedies, such as codeine, morphine, OxyContin (oxycodone) and Percodan (percocet), to name several. In terms of treatment efficacy, there is a rather large gap between the third group and the first two, in terms of both potency and invasiveness.

Nonsteroidal Antiinflammatory Drugs NSAIDs are used primarily to treat inflammation, mild to moderate pain, and fever. Specific uses include the treatment of headaches, arthritis, sports injuries, and menstrual cramps. Aspirin (also an NSAID) is used to inhibit the clotting of blood and prevent strokes and heart attacks in individuals at high risk. NSAIDs also are included in many cold and allergy preparations.

This gap gives rise to a scenario in which a treatment from the OTC/NSAID group is in insufficient to provide relief, yet the narcotic approach is undesirable due to side effects, patient preference, or simply being overkill. Narcotics carry a greater risk of physiological addiction, and may be inappropriate for patients with a predisposition or family history of addictive patterns. Further, many states tightly regulate and track prescription history on a per-physician basis, and it may be desirable to avoid being viewed as having a propensity to over prescribe or overmedicate.

While many topical treatments (creams, rubs and the like) are commercially available, conventional topical treatments fail to develop a dosage delivery medium having the delivery focus and effectiveness as in the claimed approach. Pharmaceutical developers tend to think in narrow channels, often driven by marketability and sales concerns rather than therapeutic value. Thus, a particular medication and/or administration medium may not be pursued until a sufficiently marketable demand is established, particularly when alternative approaches may assuage the same niche. In contrast, the claimed approach provides a combination of pharmaceuticals that require a prescription synergistically combined in a transdermal delivery medium as a gel form, as opposed to less effective Over-the-Counter (OTC) medications.

Pain medications typically employ one of two administrative mediums, oral and topical. Oral administration results in bloodstream absorption of the medication, thus delivering the active medicinal components in a general manner throughout the patient. An oral dosage sufficient to deliver a desired quantity of medication to an afflicted area may be unsafe in other regards because of the general bloodstream indirection of oral administration. Due to the indirectness of oral administration, it may be difficult to direct treatment to a particular afflicted area without incurring undesirable side effects elsewhere. Intravenous administration (IV) is also frequently employed, however this simply bypasses the gastrointestinal absorption into bloodstream based delivery of the medicine.

In a particular configuration, the transdermal gel for topical application of pain relief medication on a skin surface over pain afflicted tissue areas includes a synergistic combination of medicinal components (active ingredients) for musculoskeletal pain relief, such that the active ingredients have complementary properties for pain relief directed to an afflicted tissue area and have transdermal properties for absorption into the afflicted tissue area. The gel includes a liposomal base for transdermal application of the active ingredients to the afflicted area, in which the liposomal base is for suspending the active ingredients in communication with a skin surface proximate to affected tissue areas pending transdermal absorption into the affected tissue areas. A wetting agent is added for forming gel-like properties in combination with the liposomal base and the active ingredients, the gel-like properties for maintaining the active ingredients in communication with the skin, the active ingredients present in greater quantities than that which may be safely ingested for delivery to the afflicted area.

The active ingredients are deliverable in a manner to provide a concentration at the afflicted area such that an oral administration sufficient to produce a similar concentration at the afflicted areas would be unsafe. Thus, the transdermal application and delivery medium are effective for avoiding excessive dosing of oral or intravenous administration by delivering the medication directly to the afflicted area and avoiding misdirection to other areas of the patient physiology.

Neurotransmitters of pain sensations typically stimulate receptors on a receiving neuron. Types of receptors that are responsive to Glutamate include N-methyl-D-aspartate (NMDA) and alpha-amino-3-hydroxy-5-methylisoxa-zole-4-propionic acid (AMPA) receptors. These two common types of glutamate receptors are often present at the same synapse. NMDA receptors are modulated by the spinal column, and are often associated with chronic musculoskeletal pain, in contrast to other types of messengers and neurotransmitters that are modulated elsewhere in the CNS such as by the brain.

A particular configuration employs medicinal components directed to NMDA receptors. Pain is detected by two different types of peripheral nociceptor neurons, C-fiber nociceptors with slowly conducting unmyelinated axons, and A-delta nociceptors with thinly myelinated axons. During inflammation, nociceptors become sensitized, discharge spontaneously, and produce ongoing pain. Prolonged firing of C-fiber nociceptors causes release of glutamate which acts on N-methyl-D-aspartate (NMDA) receptors in the spinal cord. Activation of NMDA receptors causes the spinal cord neuron to become more responsive to all of its inputs, resulting in central sensitization. NMDA-receptor antagonists, such as ketamine, has been shown to suppress central sensitization. NMDA-receptor activation not only increases the cell's response to pain stimuli, it also decreases neuronal sensitivity to opioid receptor agonists. The claimed transdermal gel provides focused non-narcotic medication to an afflicted area in a higher concentration than that achievable orally.

In a particular arrangement, the disclosed external pain relieving medication is suitable for topical application on the epidermis (i.e. skin) and responsive to musculoskeletal ailments by providing a delivery medium for external application to an afflicted area of a patient, in which the external area is responsive to transdermal delivery. The disclosed transdermal gel includes a plurality of medicinal components having a synergistic effect for permitting musculoskeletal movement by countering the symptoms of musculoskeletal pain and being non-narcotic for avoiding dependency. In particular configurations, the medicinal components include a pain reliever directed to blocking spinal cord receptors, such as ketamine or other NMDA antagonist or blocker. Ketamine is a favored anesthetic for emergency patients with unknown medical history because it depresses breathing and circulation less than other anesthetics. The NMDA receptor antagonist dextromethorphan is one of the more commonly used cough suppressants. Lidocaine also provides similar pain reliving properties.

The transdermal gel also includes an anti-inflammatory for mitigating swelling and lymphatic responses that hinder musculoskeletal movement, such as ketoprofen and ibuprofen. Guaifenesin also has anti-inflammatory properties due to secretion thinning properties. The gel also includes a relaxant for providing flexibility and mitigating tightness of the afflicted area. Relaxant or anti-spasmodic properties are exhibited by gabapentin and baclofen. The delivery medium defined by topical application of the gel therefore provides a greater concentration of the medicinal components to the afflicted area than a concentration deliverable by oral or intravenous mechanisms. In other words, an oral or IV arrangement providing the same level of medicinal components via bloodstream delivery would require an unsafe level of medication and likely have detrimental side effects.

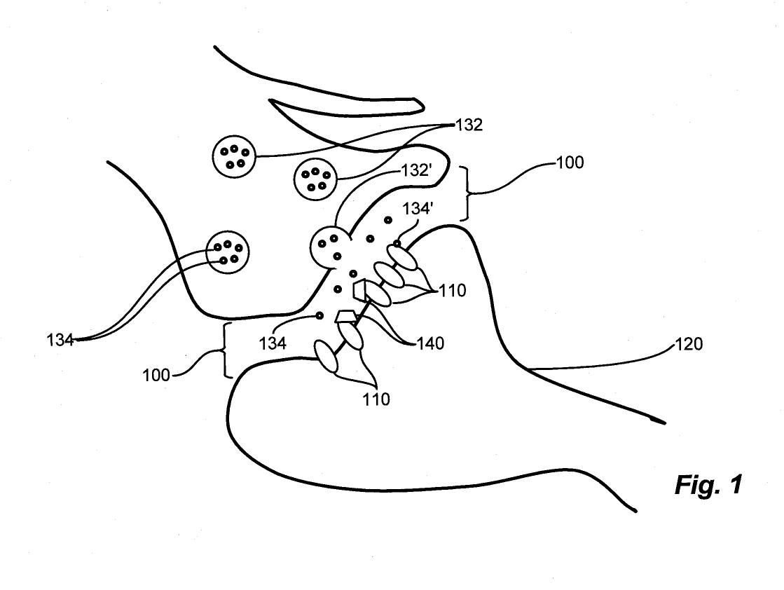

FIG. 1 is a diagram of a neural synapse 100 employing NMDA receptors 110 responsive to the present invention. Referring to FIG. 1, the synapse 100 is defined by a region, or gap, between neurons, shown in an exemplary manner as a dorsal horn neuron 120 and c-terminal 130. Synaptic vesicles 132 in the c-terminal (axon neuron) 130 contain neurotransmitters 134 that trigger a pain response. The synaptic vesicle 132′ releases neurotransmitters 134 into the synapse 100 to bind 134′ with corresponding regions of the NMDA receptor 110. NMDA receptor antagonists, or blockers 140, interfere with binding 134′ of the NMDA receptors by binding at or near receptive regions on the receptor 110 to suppress the pain stimuli.

Musculoskeletal pain typically affects the muscles, ligaments and tendons, along with the bones. Muscle tissue can be damaged with the wear and tear of daily activities. Trauma to an area, such as jerking movements, auto accidents, falls, fractures, sprains, dislocations, and direct blows to the muscle also can cause musculoskeletal pain. Other causes of pain include postural strain, repetitive movements, overuse, and prolonged immobilization.

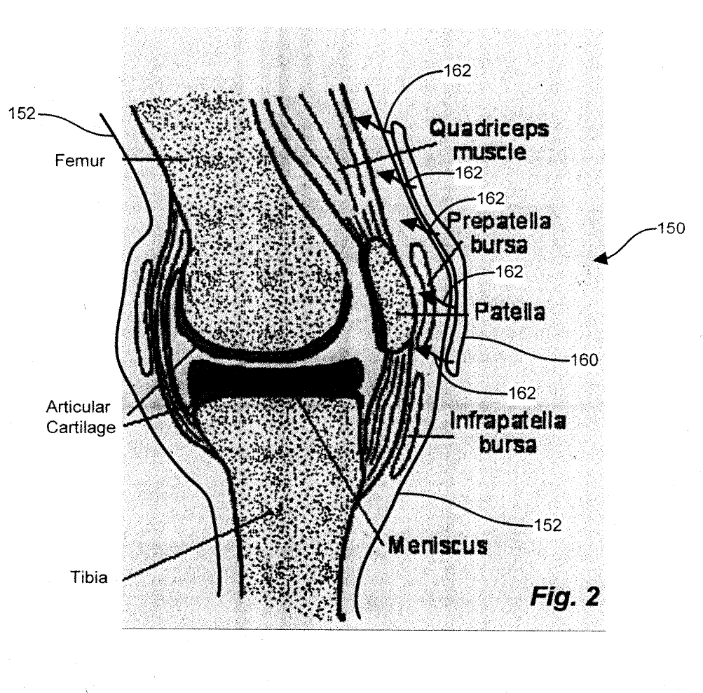

FIG. 2 is an anatomical cross section showing an epidermal region suitable for use with the transdermal gel of the present invention. The cross section 150 depicts a human knee joint and the structure of associated musculoskeletal elements responsive to the medicinal components. Application of the transdermal gel 160 on the epidermis 152 of the patient results in absorption of the medicinal components, as shown by arrows 162. Absorption by the transdermal gel provides the delivery medium for the medicinal components including the blockers 140 for suppressing the pain response.

Chronic pain can be maintained by a state of sensitization within the central nervous system that is mediated in part by the excitatory amino acids glutamate and aspartate binding to the N-methyl-D-aspartate (NMDA) receptor. A number of antagonists to the NMDA receptor are antinociceptive in animal models but are associated with significant dose-limiting side effects. Commercially available NMDA-receptor antagonists include ketamine, dextromethorphan, memantine, and amantadine. The opioids methadone, dextropropoxyphene, and ketobemidone are also antagonists at the NMDA receptor.

The NMDA receptor is known as an ionotropic receptor that allows for the transfer of electrical signals between neurons in the brain and in the spinal column. For electrical signals to pass, the NMDA receptor must be open. To remain open, an NMDA receptor must bind to glutamate and to glycine. An NMDA receptor that is bound to glycine and glutamate and has an open ion channel is called “activated.” Chemicals that deactivate the NMDA receptor are called antagonists or “blockers” because they bind to the receptor or a nearby (allosteric) site such that reception is inhibited.

The approach outlined herein is particularly effective at treatment of joint areas, such as the knee and shoulder. Joint areas are typically characterized by narrower or thinner soft tissue areas such as muscle, and tend to be dominated by denser structures such as bone, cartilage and connective tissue. Accordingly, systemic absorption into these areas is lessened, and conventional bloodstream absorption of medicinal components less effective. The disclosed approach concentrates potent amounts of active ingredients, such as ketamine, ketoprofen, lidocaine, gabapentin, guaifenesin, and baclofen directly at the receptor area, developing a potency at the treatment site unattainable by oral administration.

In a particular configuration, Ketamine has been found to be effective as an antidepressant. The relatively low but consistent transdermal delivery of Ketamine as disclosed herein provides an effective systemic infusion of Ketamine into patients exhibiting symptoms of depression, including bipolar disorder. Often, patients with depressive disorders are more susceptible to, or actively suffer from, addictive and compulsive disorders. As described above, oral administration of potent medications, particularly pain relievers and anti-spasmodics, can present triggers that exacerbate a vulnerable psychological or psychiatric condition. The transdermal administration coupled with the antidepressant effect of Ketamine achieves relief in patients who would otherwise be at increased risk for mental health issues associated with pain relief.

Accordingly, further configurations disclosed herein provide a method of providing clinical relief, including selecting active ingredients for treating pain and discomfort from among non-steroidal anti-inflamatory drug (NSAID) remedies. In many cases, the mental framework of a patient is a significant consideration in the selection of medicinal substances. Mental conditions such as an addition or predisposition to an addictive response, as well as depressive or compulsive disorders such as clinical depression, eating disorders, bipolar disorder, obsessive compulsive disorder (OCD), as well as a myriad of possible addictions, all contribute to patient makeup that warrant conservative selections of orally administered medications that may exacerbate the mental condition.

The disclosed method combining a predetermined quantity of the active ingredients, including a pain reliever, an anti-inflammatory and a relaxant and having complementary properties for pain relief of afflicted tissue and having a powder form, such that the active ingredients occurring in greater quantities than that which may be safely ingested for systemic delivery to the afflicted area. The transdermal gel is administered to an afflicted area of substantial skeletal structures, in which the substantial skeletal structures having a greater mass of skeletal and connective tissue relative to soft tissue. Generally, this refers to an afflicted area being a joint area defining a pivotal relationship between multiple linear skeletal members, such as when the pain condition results from knee or shoulder skeletal joint pain. Further, when the afflicted area is a joint area having limited bloodstream exposure for systemic absorption, the exposure to the active ingredients (medicinal components) is such that systemic levels for treating the afflicted area are outweighed by countereffects elsewhere in the patient due to general systemic absorption.

Since the administration of potent active ingredients for pain relieve can cause complications of other patient factors, selecting the active ingredients includes analyzing the patient history by identifying an existing condition of, or predisposition to, an addictive disorder, and concluding that oral administration of a safe level of quantities of the active ingredients would be ineffective for treating the afflicted area.

Alternatively, analyzing the patient history may include identifying an existing condition of, or predisposition to, an addictive disorder, and concluding that oral administration of sufficient quantities of the active ingredients for treatment would exacerbate the addictive disorder. Therefore, in patient with a high risk of substance related complications, the benefit achieved by oral administration may be offset by the detrimental mental effect, or the pain may be in an area more resilient to systemically absorbed treatment that achieving sufficiently high levels is ill-advised.

Certain active ingredients may provide, however, a beneficial mental effect. For example, Ketamine has been shown to have anti-depressant properties, as illustrated in the clinical trials below. Accordingly, analyzing a patient history for susceptibility to substance induced mental disorders, selecting the active ingredients to include an antidepressant for systemic absorption. Selection of the active ingredients therefore includes identifying a pain reliever having anti-depressant effects, in which the active ingredients include ketamine.

Analyzing the patient history may further entail diagnosing clinical depression in the patient, and diagnosing a pain condition requiring medicinal intervention. A course of treatment then selects an active ingredient having pain reliving and antidepressant properties. Selecting of a full synergistic combination of active ingredients includes a combination of: ketamine and gabapentin as a pain reliever directed to blocking spinal cord receptors, and also antidepressant properties, ketoprofen as an anti-inflammatory for mitigating swelling and lymphatic responses that hinder musculoskeletal movement, and baclofen as a relaxant and anti-spasmodic for providing flexibility and mitigating tightness of the afflicted area. Alternatively, ibuprofen may be employed instead of, or in addition to, ketoprofen and guaifenesin may be employed instead of/in addition to baclofen.

A further therapeutic effect is provided in the treatment of depression because a patient saddled with mobility limitations from pain is likely to experience compounded depressive symptoms due to their state of immobility. Increased self autonomy and self-sufficiency exemplifies the synergistic effect discussed above.

In a particular configuration, the transdermal gel is a Ketamine/Ibuprofen/Lidocaine/Gabapentin/Baclofen formulation in 10%/10%/5%/6/3% concentration, respectively. A 30 gram dose is prepared as follows in Table I:

TABLE I

| | Quantity: | |
|---|---|---|
| Active Ingredient: | | |
| Ketamine | 3 | gm |
| Ibuprofen USP | 3 | gm |
| Lidocaine USP | 1.5 | gm |
| Gabapentin | 1.8 | gm |
| Baclofen HCI, USP | 0.6 | gm |
| Wetting Agent: | | |
| Ethoxy Diglycol | 3 | mL |
| Base: | | |
| Liposomal Base | 17.3 | gm |

Preparation of the transdermal gel is as follows:
1. Measure and mix dry powder ingredients in glass mortar and Pestle until uniformly mixed.

2. Slowly incorporate ethoxy diglycol into mixture #1 until creamy consistency is formed.

3. Slowly add liposomal base in geometric dilution and bring it up to desired quantity.

4. Add Thickening agent if necessary.

5. Run gel through ointment mill to further reduce particle size of active ingredients.

6. Dispense in a jar or in an ointment tube.
Such a formulation may be expected to be viable for use for 90 days.

Conventional approaches to pain relieve have been disclosed, such as in U.S. Pat. No. 6,290,986 (Murdock '986). Murdock '986 is focused on transdermal delivery of various pharmacological agents, using a medium including lecithin organogel, as. Murdock then recites, from col. 2, line 65-col. 5, line 61, a far reaching list of substances that may be suitable for combination with lecithin organogel, and for various ailments. Murdock '986 teaches various compounds, however Murdock does not make any specific showing or teaching for pain relief. Murdock is primarily concerned with psychopharmaceutical combinations. Murdock '986 endorses the problems for which the disclosed lecithin organogel medium is directed, and addresses the problem of poor absorption for orally delivered pharmaceuticals and/or patients who benefit from delivery over an extended period or a relatively rapid delivery or higher rate of increase of plasma levels. Rather, the treatment taught by Murdock '986 is for general systemic distribution and treatment of the lecithin organogel delivered substance, to result in “acceptable plasma blood levels.” Murdock '986 therefore teaches avoidance of oral administration for providing general systemic delivery, not transdermal delivery for concentration at an afflicted area.

In the course of research and development, several case studies were undertaken in pursuit of the unexpected results of the transdermal pain gel as disclosed and claimed herein. In all of the cases disclosed below it was important to decrease or eliminate the need for conventional narcotics and analgesics, which only relieved the pain for 3-4 hours. In contrast, the disclosed Pain Gel eliminated and/or relieved pain for the patients for extended periods of time, usually 24 hours and on many occasions the pain did not return. Further, the patients remained ambulatory, in contrast to certain conventional approaches which mandate reduced activity due to sensory depravation.

Case studies concerning the disclosed approach include the following. In the studies that follow, “pain gel”, “pain ointment” refer to the transdermal compound for pain relieve as disclosed herein.

### Case #1—DX. Torn Meniscus of Right Knee:

A 69 year old woman twisted her knee and tore the Meniscus of the right knee. The symptoms were substantiated and documented on an MRI. She was treated with physical therapy but the pain continued as did her need for crutches. The pain gel was administered by the patient to the affected painful area. Patient was unable to walk without crutches, and was frequently in a wheel chair. The gel was applied q 2 hours to the affected area. Within 2 days the patient was walking without crutches and to date has been able to continue walking without the use of crutches. The ointment eliminated the pain, the inflammation was decreased and the flexibility of the. joint returned. Relief from pain and rigidity comes in less than 10 minutes after applying the gel. The Gel provided sustained (all day) pain relief that even narcotics and analgesics could not provide.

### Case #2—DX Arthritis of the Hip:

The pain gel (ointment) was applied to the left hip of a 65 year old female patient. The pain decreased and mobility returned within 5 minutes. She continues to use the pain gel, and has not needed surgery. She is mobile and without pain. The pain gel is applied periodically. Relief is consistent.

The same patient had knee replacement surgery and applied the ointment post surgery. The pain was relieved and the patient was able to walk unassisted within days of surgery.

### Case #3—DX Cervical Osteoarthritis and Neurological Pain:

A 50 year old man with persistent, night and day, neck pain secondary to osteoarthritis and bone spurs. The pain gel was the only medication that significantly reduced his dependency on narcotic analgesics.

### Case #4—DX Falling Accident:

A 55 year old woman suffered from back pain caused by trauma from a fall. She was not getting consistent relief from oral medication for pain. She used the pain gel on the affected area of the back and experienced relief within 10 minutes. She used the gel once daily until she healed.

While the system and method for pain relief using the disclosed transdermal gel has been particularly shown and described with references to embodiments thereof, it will be understood by those skilled in the art that various changes in form and details may be made therein without departing from the scope of the invention encompassed by the appended claims.

1. A method of providing clinical relief, comprising:
selecting active ingredients for treating pain and discomfort from among non-steroidal anti-inflamatory drug (NSAID) remedies;
combining a predetermined quantity of the active ingredients, including a pain reliever, an anti-inflammatory and a relaxant and having complementary properties for pain relief of afflicted tissue and having a powder form, the active ingredients occurring in greater quantities than that which may be safely ingested for systemic delivery to the afflicted area;
adding a liposomal base, the liposomal base for transdermal application of the active ingredients to the afflicted area, the liposomal base for suspending the active ingredients in communication with a skin surface proximate to afflicted tissue areas pending transdermal absorption into the afflicted tissue areas;
adding a wetting agent, the wetting agent for forming gel-like properties in combination with the liposomal base and the active ingredients, the gel-like properties for forming a transdermal gel adapted to maintain the active ingredients in communication with the skin; and
administering the transdermal gel to an afflicted area of substantial skeletal structures, the substantial skeletal structures having a greater mass of skeletal and connective tissue relative to soft tissue.

2. The method of claim 1 wherein selecting the active ingredients further comprises:
analyzing a patient history for susceptibility to substance induced mental disorders, and
selecting the active ingredients to include an antidepressant for systemic absorption.

3. The method of claim 1 wherein selecting the active ingredients further comprises identifying a pain reliever having anti-depressant effects.

4. The method of claim 3 wherein the active ingredients include Ketamine.

5. The method of claim 2 wherein analyzing the patient history further comprises
identifying an existing condition of, or predisposition to, an addictive disorder;
concluding that oral administration of a safe level of quantities of the active ingredients would be ineffective for treating the afflicted area.

6. The method of claim 2 wherein analyzing the patient history further comprises
identifying an existing condition of, or predisposition to, an addictive disorder;
concluding that oral administration of sufficient quantities of the active ingredients for treatment would exacerbate the addictive disorder.

7. The method of claim 2 wherein analyzing the patient history further comprises
diagnosing clinical depression in the patient; and
diagnosing a pain condition requiring medicinal intervention;
selecting an active ingredient having pain reliving and antidepressant properties.

8. The method of claim 7 wherein the afflicted area is a joint area defining a pivotal relationship between multiple linear skeletal members.

9. The method of claim 8 wherein the pain condition results from knee or shoulder skeletal joint pain.

10. The method of claim 7 wherein the afflicted area is a joint area having limited bloodstream exposure for systemic absorption, the exposure such that systemic levels for treating the afflicted area are outweighed by countereffects elsewhere in the patient due to general systemic absorption.

11. The method of claim 10 wherein the active ingredients further comprise:
ketamine and gabapentin as a pain reliever directed to blocking spinal cord receptors;
ketoprofen as an anti-inflammatory for mitigating swelling and lymphatic responses that hinder musculoskeletal movement; and
baclofen as a relaxant and anti-spasmodic for providing flexibility and mitigating tightness of the afflicted area.

12. A method of administering pain relieving medication responsive to musculoskeletal ailments comprising:
generating a gel based delivery medium for external application to an afflicted area of a patient, the afflicted area responsive to transdermal delivery, the delivery medium including a liposomal base and a wetting agent for forming gel-like properties;
combining a plurality of medicinal components to form a transdermal gel, the medicinal components having a synergistic effect for permitting musculoskeletal movement by countering the symptoms of musculoskeletal pain and being non-narcotic for avoiding dependency, the medicinal components including:
a pain reliever directed to blocking spinal cord receptors;
an anti-inflammatory for mitigating swelling and lymphatic responses that hinder musculoskeletal movement; and
a relaxant for providing flexibility and mitigating tightness of the afflicted area;
the delivery medium providing a greater concentration of the medicinal components to the afflicted area than a concentration deliverable by oral or intravenous mechanisms.

13. The method of claim 12 further comprising administering the transdermal gel to an afflicted area of substantial skeletal structures, the substantial skeletal structures having a greater mass of skeletal and connective tissue relative to soft tissue.

14. The method of claim 13 further comprising selecting active ingredients of the medicinal components by:
analyzing a patient history for susceptibility to substance induced mental disorders, and
selecting the active ingredients to include an antidepressant for systemic absorption.

15. The method of claim 14 wherein analyzing the patient history further comprises
identifying an existing condition of, or predisposition to, an addictive disorder;
concluding that oral administration of safe levels of the active ingredients would be insufficient for treating the afflicted area.

16. The method of claim 14 wherein analyzing the patient history further comprises
identifying an existing condition of, or predisposition to, an addictive disorder;
concluding that oral administration of sufficient quantities of the active ingredients for treatment would exacerbate the addictive disorder.

17. The method of claim 13 wherein analyzing the patient history further comprises
diagnosing clinical depression in the patient; and
diagnosing a pain condition requiring medicinal intervention;
selecting an active ingredient having pain reliving and antidepressant properties.

18. The method of claim 15 wherein the active ingredients comprise:
ketamine 10%
ibuprofen USP 10%
lidocaine USP 5%
gabapentin 6%
baclofen HCl, USP 2%; and
the wetting agent is ethoxy diglycol.

19. The method of claim 15 wherein the active ingredients comprise:
ketamine 10%
ketoprofen 20%
lidocaine USP 5%
gabapentin 6%
guaifenesin 10%; and
the wetting agent is ethoxy diglycol.

20. A method for formulating a pain relieving gel for transdermal application of pain relief medication comprising:
mixing a predetermined quantity of active ingredients, including ketamine, ibuprofen, lidocaine, gabapentin and baclofen, the active ingredients having synergistic properties for pain relief of afflicted tissue and having a powder form, the active ingredients occurring in greater quantities than that which may be safely ingested for delivery to the afflicted area;
adding a liposomal base, the liposomal base for transdermal application of the active ingredients to the afflicted area, the liposomal base for suspending the active ingredients in communication with a skin surface proximate to afflicted tissue areas pending transdermal absorption into the afflicted tissue areas; and
adding a wetting agent, the wetting agent for forming gel-like properties in combination with the liposomal base and the active ingredients, the gel-like properties for maintaining the active ingredients in communication with the skin.
